(19) United States
(12) Patent Application Publication
(10) Pub. No.: US 2007/0166379 A1
(43) Pub. Date: Jul. 19, 2007

(54) Coated tablet containing venlafaxin or its salts with controlled release

(76) Inventors: Beata Vladovicova, Hlohoec (SK); Mikulas Lehocky, Hlohovec (SK); Viera Kormanova, Hlohovec (SK)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/581,461

(22) PCT Filed: Dec. 3, 2004
(86) PCT No.: PCT/CZ04/00083

§ 371 Date: Feb. 15, 2007

(30) Foreign Application Priority Data
Dec. 3, 2003 (CZ) PV2003-3294

(51) Int. Cl. A61K 9/24

(52) U.S. Cl. 424/472

(57) ABSTRACT

A venlafaxin containing coated tablet with controlled release, which contains venlafaxin, or its salt with an inorganic or carboxylic acid, in amounts from 20 to 60 weight %, and a hydrophilic polymer in amounts from 30 to 70 weight %, based on the weight of the core, in its core, and from 1 to 3 weight % of a water-poorly permeable or impermeable polymer in its coating, and a method of preparation thereof.

### TECHNICAL FIELD

The present invention relates to a coated tablet of venlafaxin with controlled release, which is effected by combination of retardation effects in the core and in the coating of the tablet.

### BACKGROUND ART

The substance venlafaxin, of chemical name 1-[2-(dimethylamino)-1-(4-methoxyphenyl)ethyl]cyclohexanol, of formula I 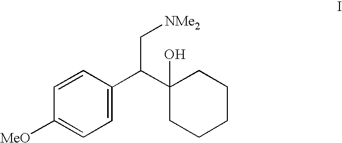
has first been described in U.S. Pat. No. 4,535,186. A venlafaxin-based preparation is used to treat depression and anxiety states.

Venlafaxin in the regularly used drug form is very quickly released into the blood stream and maximum concentrations in the blood plasma are obtained after 2 to 4 hours after administration; it is necessary to administer the drug every 6 to 8 hours. (EP 797 991). Even with such frequent administration, it is impossible to keep a constant level of drug in the blood plasma; concentration maximums and minimums always alternate. For these reasons, special attention has been devoted to development of such a drug form that would allow administering the drug once a day.

In EP patent 797 991 (line 11, p. 3), spheroids or particles of granulate are described wherein the active substance is mixed with microcrystalline cellulose (MCC) and hydroxypropyl methyl cellulose (HPMC), shaped into a spheroid, and subsequently coated with a mixture of ethyl cellulose and HPMC. A typical composition of the spheroid is 30 to 40% of venlafaxin; 50 to 70% of MCC, 0.25 to 1% of HPMC; the coating accounts for 5 to 10% of the weight. It is further stated in the cited patent that attempts to develop an ordinary type of tablet with controlled release, i.e. tablets containing a gel-forming cellulose derivative, fail. They are either physically unstable (i.e. they do not have sufficient compressibility or have capping problems) or no uniform release over 24 hours is achieved, so that they can be administered only once a day (release of the whole tablet within 8 hours is typical for this classic solution). Only the spheroid-based solution of the drug form has brought, according to the mentioned patent, the desired effect of uniform release over 24 hours.

In the following table there are typical values of release of the drug form according to EP 797 991:

| | Released |
|---|---|
| h | % |
| 2 | <30 |
| 4 | 30-55 |
| 8 | 55-80 |
| 12 | 65-90 |
| 24 | >80 |

However, production of spheroids is technologically demanding and requires special equipment. That is why efforts to achieve controlled release in the tablet have continued.

One of the possible solutions is mentioned in patent application WO 03055475. In principle, controlled release is solved by two different types of hydrophilic polymers in the tablet's core, one of which is highly viscous, while the other has low viscosity, and two different polymers in the tablet's coating, one of which is highly water-permeable and the other is purely or not at all water-permeable. An important feature of the mentioned invention includes also polyvinylpyrrolidone in the core, which is supposed to prevent reverse crystallization of venlafaxin during its release.

A typical formulation according to the cited application is in the following table:

| Item | mg |
|---|---|
| Core | |
| Venlafaxin hydrochloride | 169.7 |
| Out of which the base | 150 |
| Polyvinylpyrrolidone K30 | 150 |
| Methocel F50P | 450 |
| Methocel K100MP | 70 |
| Ludipres | 173 |
| Talc | 5 |
| Mg stearate | 2 |
| Coating | |
| HPMC Pharmacoat 606 | 22.695 |
| HPMC phthalate | 9.726 |
| Triethyl citrate | 2.598 |
| Iron oxide | 0.788 |
| Titanium dioxide | 2.373 |
| Talc | 0.324 |

The manufacture is performed by dissolving venlafaxin hydrochloride and polyvinylpyrrolidone in ethanol and spraying the solution onto Methocel F50P, which represents a low-viscosity hydrophilic polymer, in a fluidization granulator. The resulting granulate is dried and mixed with Methocel K100MP (a high-viscosity polymer), with Ludipres (lactose and polyvinylpyrrolidone) and magnesium stearate. The mixture is compressed. The tablet produced by this procedure is coated with a suspension of substances designed for the coating in a mixture of ethanol and water.

Dissolution values of thus obtained tablet, in time periods comparable with the capsules according to EP 797 991, are presented in the following table

| | Released |
|---|---|
| h | % |
| 2 | 22 |
| 4 | 39.6 |
| 8 | 60.7 |
| 12 | 74.9 |
| 24 | 95.5 |

It is apparent that thus prepared tablet succeeded in achieving a dissolution profile identical with the earlier described spheroids in the capsule.

However, disadvantages still rest in relatively complicated process of production of such tablet, high content of components in the tablet and, last but not least, the size of the tablet (weight about 1 g), which can be hard to swallow, especially for older patients.

However, it has now surprisingly turned out that it is possible to produce such a venlafaxin tablet that provides desired dissolution profiles in a simple manufacturing process and which achieves, preferably, weights lower than 0.5 g.

### DISCLOSURE OF INVENTION

The subject matter of the present invention provides a venlafaxin containing coated tablet with controlled release, which is characterized in containing, in its core, venlafaxin, or its salt with an inorganic or carboxylic acid, in amounts from 20 to 60 weight %, a hydrophilic polymer in amounts from 30 to 70 weight %, based on the weight of the core, and from 1 to 3 weight % of a water-poorly permeable or impermeable polymer in its coating.

Cellulose ester is preferably used as the hydrophilic polymer; an acrylic polymer is preferably used as the water-poorly permeable polymer.

The subject matter of the invention also includes production of tablets containing venlafaxin, or its salt with an inorganic or carboxylic acid, which is used to treat anxiety and depression. The essence of the manufacture of tablets resides in preparing a tablet material by dry mixing venlafaxin and the hydrophilic polymer, optionally with addition of colloidal silicon dioxide and magnesium stearate, followed by tabletting and adjusting the size of particles of the tablet material. The mixture is compressed into tablets. The tablet produced via this procedure is coated with an aqueous suspension of substances designed for coating, i.e. of the water-poorly permeable polymer, optionally along with talc and acetyl triethyl citrate.

The preparation of the tablet material is technologically simple, being limited only to technological steps that are not demanding with respect to energy and time. The method of preparation and choice of supplemental substances according to the invention, described herein, also ensure very good stability of the formulation and the desired physical properties of the drug form as well as the required dissolution profile identical with earlier-described venlafaxin containing capsules and tablets.

The tablet described in the present invention contains, besides the venlafaxin active substance, or its salt with an inorganic or carboxylic acid, other adjuvants, which bring about controlled release, namely a hydrophilic polymer, especially a cellulose ester, e.g. Methocel K 100M Premium EP, constituting the tablet core, and a water-poorly permeable polymer, especially an acrylic polymer, e.g. Eudragit L 30 D-55, in the tablet coating. Eudragit L 30 D-55 is a 30% aqueous dispersion of an anionic copolymer of methacrylic acid, which solubilizes at pH 5.5. At pH lower than 5 the film is not soluble and it gradually dissolves from pH above 5.5. The tablet material further comprises substances that modify flow properties of the tablet material and antiadhesive substances, which facilitate the tabletting process.

For obtaining the desired dissolution profile, the hydrophilic polymer, e.g. Methocel K 100M Premium EP, contained in the core, and the water-poorly permeable polymer, e.g. Eudragit L 30 D-55, contained in the coating, and their percentages are important according to this invention.

In a further embodiment, the tablet mixture contains substances that improve its flow properties and antiadhesive substances. The most advantageous substance for the described mixture is colloidal silicon dioxide (silica colloidalis anhydrica), preferably in amounts from 0.1 to 10%, most preferably from 1 to 5 weight %, and magnesium stearate, preferably in amounts from 0.1 to 10%, most preferably from 0.5 to 4 weight %).

The tablet material can be prepared from the above mixtures by dry mixing. For modifying the flow properties of the tablet material it is possible to prepare a briquette form the above mixture with subsequent adjustment of the particle size of the tablet material. Tablets are made from thus prepared tablet material and subsequently coated with a coating material, e.g. Eudragit L 30 D-55, preferably in amounts from 1 to 3 weight %.

The process of production of the tablet material, cores and coated tablets described herein, as well as selection of adjuvants according to this invention allow to prepare a tablet material and solid drug forms with excellent physical parameters and required dissolution profile.

### EXAMPLES

### Example 1

### Venlafaxin 75 mg Retard Tablets

| Starting materials | Weight in g |
|---|---|
| Venlafaxin hydrochloride | 0.0848400 |
| Methocel K 100M Premium EP | 0.1750000 |
| Magnesium stearate | 0.0050000 |
| Colloidal silicon dioxide | 0.0081600 |
| Total weight of the core in g: | 0.2730000 |
| Eudragit L 30 D-55 (30% aqueous dispersion) | 0.0200000 |
| (corresponds to the dry matter) | (0.006000) |
| Acetyl triethyl citrate | 0.0012000 |
| Talc | 0.0020000 |
| Total weight of the tablet in g | 0.2822000 |

A description of the technology of tablet preparation
1. Mixing I: the active substance and Methocel K 100M Premium EP are mixed in a homogenizing device for 15 minutes.
2. Mixing II: the final treatment is added—silicii dioxidum colloidale and magnesii stearas are mixed in the homogenizing device for 15 minutes.
3. Preparation of the briquette
4. Grinding—adjustment of the particle size of the tablet material
5. Tabletting
6. Coating with aqueous suspension of Eudragit, talc and acetyl triethyl citrate.

For tablets with controlled release, the dissolution profile is an important variable. The dissolution profile of tablets produced via this procedure is in a very good agreement with the already registered and sold formulation Trevilor retard 75 mg and 150 mg, resp., of Wyeth-Pharma GmbH. The dissolution profile was measured using a standard procedure. Dissolutions of Venlafaxin 75 mg retard tablets in time intervals comparable with capsules Trevilor are presented in the following table.

| | Released % of the active | Released % of the active |
|---|---|---|
| | substance Trevilor | substance Vennlafaxin |
| Hrs | retard 75 mg capsules | 75 mg retard tablets |
| 2 | 15 | 19 |
| 4 | 45 | 45 |
| 8 | 76 | 75 |
| 12 | 88 | 88 |

### Example 2

Venlafaxin 150 mg was obtained by the same procedure.

### Venlafaxin 150 mg Retard Tablets

| Starting materials | Weight in g |
|---|---|
| Venlafaxin hydrochloride | 0.1697000 |
| Methocel K 100M Premium EP | 0.2000000 |
| Magnesium stearate | 0.0100000 |
| Colloidal silicon dioxide | 0.0163000 |
| Total weight of the core in g: | 0.3960000 |
| Eudragit L 30 D-55 (30% aqueous dispersion) | 0.0200000 |
| (corresponds to the dry matter) | (0.006000) |
| Acetyl triethyl citrate | 0.0012000 |
| Talc | 0.0020000 |
| Total weight of the tablet in g | 0.4052000 |

Dissolutions of Venlafaxin 150 mg retard tablets in time intervals comparable with capsules Trevilor are presented in the following table.

| | Released % of the active | Released % of the active |
|---|---|---|
| | substance Trevilor | substance Venlafaxin |
| Hrs | retard 150 mg capsules | 150 mg retard tablets |
| 2 | 16 | 26 |
| 4 | 42 | 49 |
| 8 | 70 | 76 |
| 12 | 81 | 89 |

It has turned out therefore that the tablets produced by the present new method have identical prosperities as the already sold formulations, their production is simple, and at the same time, tablets amount to less than 0.5 g in weight.

1. A coated tablet with controlled release, wherein the coated tablet comprises venlafaxin, or its salt with an inorganic or carboxylic acid, in amounts from 20 to 60 weight %, and a hydrophilic polymer in amounts from 30 up to 70 weight %, based on the weight of the core, in its core, and from 1 up to 3 weight % of a water-poorly permeable or impermeable polymer in its coating.

2. The coated tablet according to claim 1, wherein the total weight of the coated tablet does not exceed 500 mg.

3. The coated tablet according to claim 1, wherein the hydrophilic polymer comprises a cellulose ester.

4. The coated tablet of claim 1, wherein the coated tablet comprises the water-poorly permeable polymer, and wherein the water-poorly permeable polymer comprises an acrylic polymer.

5. The coated tablet of claim 1, wherein the coated tablet comprises 75 mg of the active substance, based on the venlafaxin base, from 100 to 200 mg of Methocel K 100M Premium EP in its core and from 3 to 10 mg of Eudragit L 30 D-55 in its coating.

6. The coated tablet of claim 1, wherein the coated tablet comprises 150 mg of the active substance, based on the venlafaxin base, from 150 to 300 mg of Methocel K 100M Premium EP in its core and from 3 to 10 mg of Eudragit L 30 D in its coating.

7. A method of production of the tablet according to claim 1, comprising
mixing a mixture of venlafaxin and a hydrophilic polymer in a homogenizing device,
optionally, subsequently adding colloidal silicon dioxide along with magnesium stearate,
homogenizing the mixture to form a blend,
briguetting the blend to form a tablet material which comprises a particle size,
adjusting the particle size of the tablet material,
compressing the tablet material into tablets, and
coating the tablets with a suspension,
wherein the suspension comprises the water-poorly permeable polymer and, optionally, talc and acetyl triethyl citrate.

8. The coated tablet of claim 1, wherein the coated tablet comprises, in its coating, the water-poorly permeable polymer.

9. The coated tablet of claim 1, wherein the coated tablet comprises, in its coating, the impermeable polymer.

10. The method of claim 7, wherein the colloidal silicon dioxide along with magnesium state are subsequently added.

11. The method of claim 7, wherein the suspension comprises the water-poorly permeable polymer, talc, and triethyl citrate.

12. The coated tablet of claim 2, wherein the hydrophilic polymer comprises a cellulose ester.

13. The coated tablet of claim 2, wherein the coated tablet comprises the water-poorly permeable polymer, and wherein the water-poorly permeable polymer comprises an acrylic polymer.

14. The coated tablet of claim 3, wherein the coated tablet comprises the water-poorly permeable polymer, and wherein the water-poorly permeable polymer comprises an acrylic polymer.

15. The coated tablet of claim 2, wherein the coated tablet comprises 75 mg of the active substance, based on the venlafaxin base, from 100 to 200 mg of Methocel K 100M Premium EP in its core and from 3 to 10 mg of Eudragit L 30 D-55 in its coating.

16. The coated tablet of claim 3, wherein the coated tablet comprises 75 mg of the active substance, based on the venlafaxin base, from 100 to 200 mg of Methocel K 100M Premium EP in its core and from 3 to 10 mg of Eudragit L 30 D-55 in its coating.

17. The coated tablet of claim 4, wherein the coated tablet comprises 75 mg of the active substance, based on the venlafaxin base, from 100 to 200 mg of Methocel K 100M Premium EP in its core and from 3 to 10 mg of Eudragit L 30 D-55 in its coating.

18. The coated tablet of claim 2, wherein the coated tablet comprises 150 mg of the active substance, based on the venlafaxin base, from 150 to 300 mg of Methocel K 100M Premium EP in its core and from 3 to 10 mg of Eudragit L 30 D in its coating.

19. The coated tablet of claim 3, wherein the coated tablet comprises 150 mg of the active substance, based on the venlafaxin base, from 150 to 300 mg of Methocel K 100M Premium EP in its core and from 3 to 10 mg of Eudragit L 30 D in its coating.

20. The coated tablet of claim 4, wherein the coated tablet comprises 150 mg of the active substance, based on the venlafaxin base, from 150 to 300 mg of Methocel K 100M Premium EP in its core and from 3 to 10 mg of Eudragit L 30 D in its coating.
